(19) United States
(12) Patent Application Publication
(10) Pub. No.: US 2026/0048096 A1
(43) Pub. Date: Feb. 19, 2026

(54) METHODS AND COMPOSITIONS FOR PRESERVING THE RETINAL OUTER NUCLEAR LAYER (ONL), PHOTORECEPTORS, AND RETINAL THICKNESS

(71) Applicant: Stealth BioTherapeutics Inc., Needham, MA (US)

(72) Inventors: David Brown, Needham, MA (US); Kristy Vardy, Needham, MA (US); James Wakefield, Needham, MA (US)

(73) Assignee: Stealth BioTherapeutics Inc., Needham, MA (US)

(21) Appl. No.: 19/301,213

(22) Filed: Aug. 15, 2025

Related U.S. Application Data
(60) Provisional application No. 63/684,687, filed on Aug. 19, 2024.
(60) Provisional application No. 63/743,585, filed on Jan. 9, 2025.

(51) Int. Cl. A61K 38/06; A61K 45/06; A61P 27/02

(52) U.S. Cl. CPC A61K 38/06; A61K 45/06; A61P 27/02

(57) ABSTRACT

The present disclosure relates to compositions, methods and related uses for preventing, inhibiting, ameliorating or delaying (slowing): (i) the thinning of the retinal outer nuclear layer (ONL), and/or (ii) reduction in total retinal thickness in a subject. The present disclosure further relates generally to compositions, methods and related uses for protecting: (i) the retinal outer nuclear layer (ONL) and/or the retina from light- or age-induced damage, and/or (ii) photoreceptors from light- or age-induced damage. In particular, the present technology relates to administering bevemipretide, or a pharmaceutically acceptable salt, stereoisomer, tautomer, hydrate, and/or solvate thereof to subjects in need thereof.

### CROSS-REFERENCE TO RELATED APPLICATIONS

This application claims the benefit of and priority to U.S. Provisional Patent Application No. 63/684,687, filed Aug. 19, 2024, and U.S. Provisional Patent Application No. 63/743,585, filed Jan. 9, 2025, the contents of which are incorporated herein by reference in their entireties for any and all purposes.

### TECHNICAL FIELD

The present technology relates generally to compositions, related uses, and methods for preventing, inhibiting, ameliorating, or delaying (slowing): (i) the thinning of the retinal outer nuclear layer (ONL), and/or (ii) reduction in the total retinal thickness in a mammalian subject. The present technology further relates generally to compositions and methods for protecting: (i) the retinal outer nuclear layer (ONL) and/or the retina from light- or age-induced damage, and/or (ii) photoreceptors from light- or age-induced damage. In particular, the present technology relates to administering bevemipretide (a.k.a., (R)-2-amino-N—((S)-1-(((S)-5-amino-1-(3-benzyl-1,2,4-oxadiazol-5-yl)pentyl)amino)-3-(4-hydroxy-2,6-dimethylphenyl)-1-oxopropan-2-yl)-5-guanidinopentanamide, chemical structure shown below), or a pharmaceutically acceptable salt, stereoisomer, tautomer, hydrate, and/or solvate thereof to subjects in need thereof.

### INTRODUCTION

The following description is provided to assist the understanding of the reader. None of the information provided or references cited is admitted as being prior art to the present technology.

The vertebral retina is adapted to capturing light photons and transmitting this information to other structures in the central nervous system. The neurosensory retina can be divided into several layers. The retinal outer nuclear layer (ONL) contains the nuclei of the photoreceptor cells. Thinning of the ONL and/or reductions in total retinal thickness is an irreversible phenomenon implicated in a number of disease conditions, which affect millions of individuals worldwide. The ONL (which comprises photoreceptor cell bodies) is subject to light- and age-induced damage. As such, there is a need for methods, compounds, and compositions for preventing and/or delaying (slowing) the progression of ONL thinning, reduction of total retinal thickness, protecting the ONL from light- or age-induced damage and/or protecting photoreceptors from light- or age-induced damage.

### SUMMARY

In one aspect, the present disclosure provides a method for preventing, inhibiting, ameliorating, or delaying thinning of the retinal outer nuclear layer (ONL) in a subject in need thereof, the method comprising administering to the subject a therapeutically effective amount of bevemipretide, or a pharmaceutically acceptable salt, stereoisomer, tautomer, hydrate, and/or solvate thereof. In some embodiments, the subject is a mammal. In some embodiments, the subject is a human. In some embodiments, the bevemipretide, or a pharmaceutically acceptable salt, stereoisomer, tautomer, hydrate, and/or solvate thereof is administered topically. In some embodiments, the bevemipretide, or a pharmaceutically acceptable salt, stereoisomer, tautomer, hydrate, and/or solvate thereof is administered subcutaneously. In some embodiments, the bevemipretide, or a pharmaceutically acceptable salt, stereoisomer, tautomer, hydrate, and/or solvate thereof is administered intraocularly. In some embodiments, the bevemipretide, or a pharmaceutically acceptable salt, stereoisomer, tautomer, hydrate, and/or solvate thereof is administered ophthalmically. In some embodiments, the bevemipretide, or a pharmaceutically acceptable salt, stereoisomer, tautomer, hydrate, and/or solvate thereof is administered orally, intranasally, systemically, intravenously, intraperitoneally, intradermally, intrathecally, intracerebroventricularly, iontophoretically, transmucosally, or intramuscularly. In some embodiments, the bevemipretide, or a pharmaceutically acceptable salt, stereoisomer, tautomer, hydrate, and/or solvate thereof is administered daily for 2 weeks or more, 12 weeks or more, 24 weeks or more, 52 weeks or more, or 2 years or more. In some embodiments, the bevemipretide, or a pharmaceutically acceptable salt, stereoisomer, tautomer, hydrate, and/or solvate thereof is administered via eye drops comprising the bevemipretide, or a pharmaceutically acceptable salt, stereoisomer, tautomer, hydrate, and/or solvate thereof. In some embodiments, the eye drops comprise the bevemipretide, or a pharmaceutically acceptable salt, stereoisomer, tautomer, hydrate, and/or solvate thereof in a concentration of about 0.2% to about 5% (wt./vol.) inclusive, about 1% to about 5% (wt./vol.) inclusive, about 1% to about 3% (wt./vol.) inclusive or about 0.5% to about 2% (wt./vol.) inclusive. In some embodiments, the eye drops comprise the bevemipretide, or a pharmaceutically acceptable salt, stereoisomer, tautomer, hydrate, and/or solvate thereof in a concentration of about 0.5% (wt./vol.), about 1% (wt./vol.), about 1.5% (wt./vol.), about 2% (wt./vol.), about 2.5% (wt./vol.), or about 3% (wt./vol). Formulation of eye drops is typically calculated using the free-base mass of bevemipretide when determining the weight (wt.) to volume (vol.) ratio regardless of whether or not a salt, hydrate and/or solvate is used but proper adjustments are made to the calculations to ensure potency. In some embodiments, the method further comprises separately, sequentially, or simultaneously administering an additional treatment to the subject. In some embodiments, the additional treatment comprises administering an AREDS or AREDS 2 vitamin formula to the subject. In some embodiments, the additional treatment comprises administering an antioxidant, a metal complexer, an anti-inflammatory drug, an antibiotic, a mast cell stabilizer (e.g., an inhibitor of mast cell degranulation), and/or an antihistamine to the subject. In some embodiments, the antioxidant is vitamin A, vitamin C, vitamin E, lycopene, selenium, α-lipoic acid, coenzyme Q, glutathione, or a carotenoid. In some embodiments, the additional treatment comprises administering mometasone furoate, tacrolimus, quercetin, or diphenhydramine to the subject. In some embodiments, the additional treatment comprises administering a flavonoid, a coumarin, a phenol or a terpenoid to the subject. In some embodiments, a flavonoid, a coumarin, a phenol, or a terpenoid is administered to the subject as the inhibitor of mast cell degranulation. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9 (1aH)-one), sinomenine, indoline (2,3-dihydro-1H-indole) or xestospongin C([1R-(1R,4aR,11R,12aS,13S,16aS,23R,24aS)]-eicosahydro-5H,17H-1,23:11,13-diethano-2H, 14H-[1,11]dioxacycloeicosino[2,3-b:12,13-b1]dipyridine). In some embodiments, the additional treatment comprises administering to the subject a therapeutic agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof. In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the bevemipretide administered is formulated from a tris-HCl salt, a bis-HCl salt, or a mono-HCl salt. In some embodiments, the bevemipretide administered is formulated from the tris-HCl salt of formula:

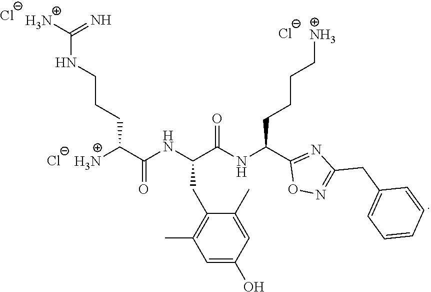 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

In another aspect, the present disclosure provides a method for preventing, inhibiting, ameliorating, or delaying reduction in total retinal thickness in a subject in need thereof, the method comprising administering to the subject a therapeutically effective amount of bevemipretide, or a pharmaceutically acceptable salt, stereoisomer, tautomer, hydrate, and/or solvate thereof. In some embodiments, the subject is a mammal. In some embodiments, the subject is a human. In some embodiments, the bevemipretide, or a pharmaceutically acceptable salt, stereoisomer, tautomer, hydrate, and/or solvate thereof is administered topically. In some embodiments, the bevemipretide, or a pharmaceutically acceptable salt, stereoisomer, tautomer, hydrate, and/or solvate thereof is administered subcutaneously. In some embodiments, the bevemipretide, or a pharmaceutically acceptable salt, stereoisomer, tautomer, hydrate, and/or solvate thereof is administered intraocularly. In some embodiments, the bevemipretide, or a pharmaceutically acceptable salt, stereoisomer, tautomer, hydrate, and/or solvate thereof is administered ophthalmically. In some embodiments, the bevemipretide, or a pharmaceutically acceptable salt, stereoisomer, tautomer, hydrate, and/or solvate thereof is administered orally, intranasally, systemically, intravenously, intraperitoneally, intradermally, intrathecally, intracerebroventricularly, iontophoretically, transmucosally, or intramuscularly. In some embodiments, the bevemipretide, or a pharmaceutically acceptable salt, stereoisomer, tautomer, hydrate, and/or solvate thereof is administered daily for 2 weeks or more, 12 weeks or more, 24 weeks or more, 52 weeks or more, or 2 years or more. In some embodiments, the bevemipretide, or a pharmaceutically acceptable salt, stereoisomer, tautomer, hydrate, and/or solvate thereof is administered via eye drops comprising the bevemipretide, or a pharmaceutically acceptable salt, stereoisomer, tautomer, hydrate, and/or solvate thereof. In some embodiments, the eye drops comprise the bevemipretide, or a pharmaceutically acceptable salt, stereoisomer, tautomer, hydrate, and/or solvate thereof in a concentration of about 0.2% to about 5% (wt./vol.) inclusive, about 1% to about 5% (wt./vol.) inclusive, about 1% to about 3% (wt./vol.) inclusive or about 0.5% to about 2% (wt./vol.) inclusive. In some embodiments, the eye drops comprise the bevemipretide, or a pharmaceutically acceptable salt, stereoisomer, tautomer, hydrate, and/or solvate thereof in a concentration of about 0.5% (wt./vol.), about 1% (wt./vol.), about 1.5% (wt./vol.), about 2% (wt./vol.), about 2.5% (wt./vol.), or about 3% (wt./vol). Formulation of eye drops is typically calculated using the free-base mass of bevemipretide when determining the weight (wt.) to volume (vol.) ratio regardless of whether or not a salt, hydrate or solvate is used but proper adjustments are made to the calculations to ensure potency. In some embodiments, the method further comprises separately, sequentially, or simultaneously administering an additional treatment to the subject. In some embodiments, the additional treatment comprises administering an AREDS or AREDS 2 vitamin formula to the subject. In some embodiments, the additional treatment comprises administering an antioxidant, a metal complexer, an anti-inflammatory drug, an antibiotic, a mast cell stabilizer, and/or an antihistamine to the subject. In some embodiments, the antioxidant is vitamin A, vitamin C, vitamin E, lycopene, selenium, α-lipoic acid, coenzyme Q, glutathione, or a carotenoid. In some embodiments, the additional treatment comprises administering mometasone furoate, tacrolimus, quercetin, or diphenhydramine to the subject. In some embodiments, the additional treatment comprises administering a flavonoid, a coumarin, a phenol or a terpenoid. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2/2)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9 (1aH)-one), sinomenine, indoline (2,3-dihydro-1H-indole) or xestospongin C([1R-(1R,4aR,11R,12aS,13S,16aS,23R,24aS)]-eicosahydro-5H,17H-1,23:11,13-diethano-2H, 14H-[1,11]dioxacycloeicosino[2,3-b:12,13-b1]dipyridine). In some embodiments, the additional treatment comprises administering to the subject a therapeutic agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof. In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the bevemipretide administered is formulated from a tris-HCl salt, a bis-HCl salt, or a mono-HCl salt. In some embodiments, the bevemipretide administered is formulated from the tris-HCl salt of formula:

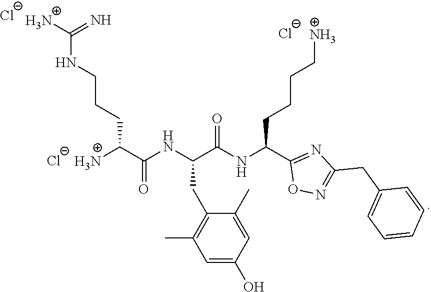 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

In another aspect, the present disclosure provides a method for protecting the retinal outer nuclear layer (ONL) and/or the retina from light- or age-induced damage in a subject in need thereof, the method comprising administering to the subject a therapeutically effective amount of bevemipretide, or a pharmaceutically acceptable salt, stereoisomer, tautomer, hydrate, and/or solvate thereof. In some embodiments, the subject is a mammal. In some embodiments, the subject is a human. In some embodiments, the bevemipretide, or a pharmaceutically acceptable salt, stereoisomer, tautomer, hydrate, and/or solvate thereof is administered topically. In some embodiments, the bevemipretide, or a pharmaceutically acceptable salt, stereoisomer, tautomer, hydrate, and/or solvate thereof is administered subcutaneously. In some embodiments, the bevemipretide, or a pharmaceutically acceptable salt, stereoisomer, tautomer, hydrate, and/or solvate thereof is administered intraocularly. In some embodiments, the bevemipretide, or a pharmaceutically acceptable salt, stereoisomer, tautomer, hydrate, and/or solvate thereof is administered ophthalmically. In some embodiments, the bevemipretide, or a pharmaceutically acceptable salt, stereoisomer, tautomer, hydrate, and/or solvate thereof is administered orally, intranasally, systemically, intravenously, intraperitoneally, intradermally, intrathecally, intracerebroventricularly, iontophoretically, transmucosally, or intramuscularly. In some embodiments, the bevemipretide, or a pharmaceutically acceptable salt, stereoisomer, tautomer, hydrate, and/or solvate thereof is administered daily for 2 weeks or more, 12 weeks or more, 24 weeks or more, 52 weeks or more, or 2 years or more. In some embodiments, the bevemipretide, or a pharmaceutically acceptable salt, stereoisomer, tautomer, hydrate, and/or solvate thereof is administered via eye drops comprising the bevemipretide, or a pharmaceutically acceptable salt, stereoisomer, tautomer, hydrate, and/or solvate thereof. In some embodiments, the eye drops comprise the bevemipretide, or a pharmaceutically acceptable salt, stereoisomer, tautomer, hydrate, and/or solvate thereof in a concentration of about 0.2% to about 5% (wt./vol.) inclusive, about 1% to about 5% (wt./vol.) inclusive, about 1% to about 3% (wt./vol.) inclusive or about 0.5% to about 2% (wt./vol.) inclusive. In some embodiments, the eye drops comprise the bevemipretide, or a pharmaceutically acceptable salt, stereoisomer, tautomer, hydrate, and/or solvate thereof in a concentration of about 0.5% (wt./vol.), about 1% (wt./vol.), about 1.5% (wt./vol.), about 2% (wt./vol.), about 2.5% (wt./vol.), or about 3% (wt./vol). Formulation of eye drops is typically calculated using the free-base mass of bevemipretide when determining the weight (wt.) to volume (vol.) ratio regardless of whether or not a salt, hydrate or solvate is used but proper adjustments are made to the calculations to ensure potency. In some embodiments, the method further comprises separately, sequentially, or simultaneously administering an additional treatment to the subject. In some embodiments, the additional treatment comprises administering an AREDS or AREDS 2 vitamin formula to the subject. In some embodiments, the additional treatment comprises administering an antioxidant, a metal complexer, an anti-inflammatory drug, an antibiotic, a mast cell stabilizer, and/or an antihistamine to the subject. In some embodiments, the antioxidant is vitamin A, vitamin C, vitamin E, lycopene, selenium, α-lipoic acid, coenzyme Q, glutathione, or a carotenoid. In some embodiments, the additional treatment comprises administering mometasone furoate, tacrolimus, quercetin, or diphenhydramine to the subject. In some embodiments, the additional treatment comprises administering a flavonoid, a coumarin, a phenol, or a terpenoid to the subject. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9 (1aH)-one), sinomenine, indoline (2,3-dihydro-1H-indole) or xestospongin C([1R-(1R,4aR,11R,12aS,13S,16aS,23R,24aS)]-eicosahydro-5H, 17H-1,23:11,13-diethano-2H, 14H-[1,11]dioxacycloeicosino[2,3-b:12,13-b1]dipyridine). In some embodiments, the additional treatment comprises administering to the subject a therapeutic agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof. In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the bevemipretide administered is formulated from a tris-HCl salt, a bis-HCl salt, or a mono-HCl salt. In some embodiments, the bevemipretide administered is formulated from the tris-HCl salt of formula:

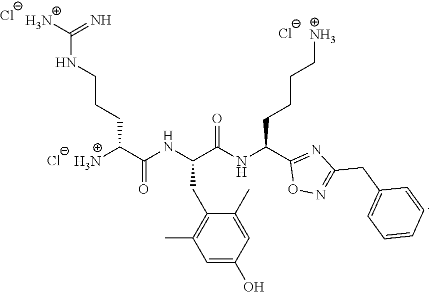 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

In another aspect, the present disclosure provides, a method for protecting photoreceptors from light- or age-induced damage in a subject in need thereof, the method comprising administering to the subject a therapeutically effective amount of bevemipretide, or a pharmaceutically acceptable salt, stereoisomer, tautomer, hydrate, and/or solvate thereof. In some embodiments, the subject is a mammal. In some embodiments, the subject is a human. In some embodiments, the bevemipretide, or a pharmaceutically acceptable salt, stereoisomer, tautomer, hydrate, and/or solvate thereof is administered topically, subcutaneously, intraocularly, or ophthalmically. In some embodiments, the bevemipretide, or a pharmaceutically acceptable salt, stereoisomer, tautomer, hydrate, and/or solvate thereof is administered orally, intranasally, systemically, intravenously, intraperitoneally, intradermally, intrathecally, intracerebroventricularly, iontophoretically, transmucosally, or intramuscularly. In some embodiments, the bevemipretide, or a pharmaceutically acceptable salt, stereoisomer, tautomer, hydrate, and/or solvate thereof is administered daily for 2 weeks or more, 12 weeks or more, 24 weeks or more, 52 weeks or more, or 2 years or more. In some embodiments, the bevemipretide, or a pharmaceutically acceptable salt, stereoisomer, tautomer, hydrate, and/or solvate thereof is administered via eye drops comprising the bevemipretide, or a pharmaceutically acceptable salt, stereoisomer, tautomer, hydrate, and/or solvate thereof. In some embodiments, the eye drops comprise the bevemipretide, or a pharmaceutically acceptable salt, stereoisomer, tautomer, hydrate, and/or solvate thereof in a concentration of about 0.2% to about 5% (wt./vol.) inclusive, about 1% to about 5% (wt./vol.) inclusive, about 1% to about 3% (wt./vol.) inclusive or about 0.5% to about 2% (wt./vol.) inclusive. In some embodiments, the eye drops comprise the bevemipretide, or a pharmaceutically acceptable salt, stereoisomer, tautomer, hydrate, and/or solvate thereof in a concentration of about 0.5% (wt./vol.), about 1% (wt./vol.), about 1.5% (wt./vol.), about 2% (wt./vol.), about 2.5% (wt./vol.), or about 3% (wt./vol). Formulation of eye drops is typically calculated using the free-base mass of bevemipretide when determining the weight (wt.) to volume (vol.) ratio regardless of whether or not a salt, hydrate or solvate is used but proper adjustments are made to the calculations to ensure potency. In some embodiments, the method further comprises separately, sequentially, or simultaneously administering an additional treatment to the subject. In some embodiments, the additional treatment comprises administering an AREDS or AREDS 2 vitamin formula to the subject. In some embodiments, the additional treatment comprises administering an antioxidant, a metal complexer, an anti-inflammatory drug, an antibiotic, a mast cell stabilizer, and/or an antihistamine to the subject. In some embodiments, the antioxidant is vitamin A, vitamin C, vitamin E, lycopene, selenium, α-lipoic acid, coenzyme Q, glutathione, or a carotenoid. In some embodiments, the additional treatment comprises administering mometasone furoate, tacrolimus, quercetin, or diphenhydramine to the subject. In some embodiments, the additional treatment comprises administering a flavonoid, a coumarin, a phenol, or a terpenoid to the subject. In some embodiments, the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin. In some embodiments, the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2E)-3-phenylprop-2-enoic acid) or ellagic acid (2,3,7,8-tetrahydroxy[1]benzopyrano[5,4,3-cde][1]benzopyran-5,10-dione). In some embodiments, the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one). In some embodiments, the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9 (1aH)-one), sinomenine, indoline (2,3-dihydro-1H-indole) or xestospongin C([1R-(1R,4aR,11R,12aS,13S,16aS,23R,24aS)]-eicosahydro-5H, 17H-1,23:11,13-diethano-2H,14H-[1,11]dioxacycloeicosino[2,3-b:12,13-b1]dipyridine). In some embodiments, the additional treatment comprises administering to the subject a therapeutic agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof. In some embodiments, the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt. In some embodiments, the bevemipretide administered is formulated from a tris-HCl salt, a bis-HCl salt, or a mono-HCl salt. In some embodiments, the bevemipretide administered is formulated from the tris-HCl salt of formula:

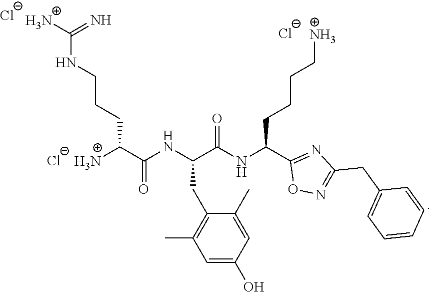 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

In some embodiments, the photoreceptors are protected from light-induced damage. In some embodiments, the photoreceptors are protected from age-induced damage.

In another aspect, the present disclosure provides a composition, formulation, or medicament comprising a therapeutically effective amount of bevemipretide for use in preventing, inhibiting, ameliorating, or delaying thinning of the retinal outer nuclear layer (ONL) in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides a composition, formulation, or medicament comprising a therapeutically effective amount of bevemipretide for use in preventing, inhibiting, ameliorating, or delaying reduction in total retinal thickness in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides a composition formulation, or medicament comprising a therapeutically effective amount of bevemipretide for use in protecting the retinal outer nuclear layer (ONL) and/or the retina from light- or age-induced damage in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides a composition, formulation, or medicament comprising a therapeutically effective amount of bevemipretide for use in protecting photoreceptors from light- or age-induced damage in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides use of a composition, formulation, or medicament comprising a therapeutically effective amount of bevemipretide in preventing, inhibiting, ameliorating, or delaying thinning of the retinal outer nuclear layer (ONL) in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides use of a composition, formulation, or medicament comprising a therapeutically effective amount of bevemipretide in preventing, inhibiting, ameliorating, or delaying reduction in total retinal thickness in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides use of a composition, formulation, or medicament comprising a therapeutically effective amount of bevemipretide in protecting the retinal outer nuclear layer (ONL) and/or the retina from light- or age-induced damage in a subject in need thereof, wherein the subject is administered the composition, formulation, or medicament comprising bevemipretide, or a pharmaceutically acceptable salt, stereoisomer, tautomer, hydrate, and/or solvate thereof.

In another aspect, the present disclosure provides use of a composition, formulation, or medicament comprising a therapeutically effective amount of bevemipretide in protecting photoreceptors from light- or age-induced damage in a subject in need thereof, wherein the subject is administered the composition, formulation, or medicament comprising bevemipretide, or a pharmaceutically acceptable salt, stereoisomer, tautomer, hydrate, and/or solvate thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are OCT images showing the baseline outer nuclear layer (ONL) (FIG. 1A) and the ONL following retinal damage (FIG. 1B) in a bright light-induced retinal degeneration rat model.

FIGS. 1C and 1D are charts showing the change in outer nuclear layer (ONL) thickness (FIG. 1C) and total retinal thickness (FIG. 1D) over time in rats treated with vehicle or bevemipretide before and after exposure to bright light-induced retinal damage.

FIG. 1E is a series of images showing immunohistochemical staining of retinal tissues isolated from vehicle- and bevemipretide-treated rats two weeks after bright light-induced retinal damage. Tissues were stained for retinal pigment epithelial (RPE) cells (RPE65), rod photoreceptors (rhodopsin), and nuclei (DAPI) and analyzed by fluorescent microscopy.

FIGS. 1F and 1G are bar charts showing a trend for increased Rhodopsin (FIG. 1F) and increased RPE65 (FIG. 1G) signal for eyes treated with bevemipretide as compared to vehicle-treated eyes in rats after exposure to bright light-induced retinal damage.

FIGS. 1H and 11 are an image and bar chart, respectively, showing RPE loss in vehicle-treated and bevemipretide-treated eyes in rats after exposure to bright light-induced retinal damage. Vehicle-treated eyes had a significantly higher % loss of RPE cells as compared to eyes treated with bevemipretide (p=0.0007).

FIGS. 1J and 1K are an image and a bar chart, respectively, showing photoreceptor loss in vehicle-treated and bevemipretide-treated eyes in rats after exposure to bright light-induced retinal damage. Vehicle-treated eyes had a significantly higher % loss of photoreceptors as compared to eyes treated with bevemipretide (p=0.0008).

FIGS. 2A and 2B are charts showing day 29 tissue and plasma concentrations of bevemipretide 24 hours after final dose in New Zealand white rabbits (1% or 2% QD) and Yucatan minipigs (5% BID). New Zealand white rabbits and Yucatan minipigs were administered bevemipretide eyedrops (concentrations up to 5% BID OU) for 28 days. Blood samples were collected and both eyes were harvested following euthanasia at 24 hours after final dose. Plasma and eye tissue concentrations were analyzed by LC-MS/MS (LLOQ: 0.1-1 ng/ml; 0.2-5 ng/g). PK parameters were calculated using noncompartmental analysis (Phoenix WinNonlin).

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, NY, 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

### Definitions

The definitions of certain terms as used in this specification are provided below. Unless defined otherwise, all technical and scientific terms used herein generally have the same meaning as commonly understood by one of ordinary skill in the art to which the present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administrating” or “administration” of an agent (i.e., a therapeutic agent) or compound/drug product (including a composition (i.e., a formulation or medicament)) to a subject includes any route of introducing or delivering to a subject a compound, composition or formulation to perform its intended function. Administration can be carried out by any suitable route, such as oral administration. Administration can be carried out subcutaneously. Administration can be carried out topically. Administration can be carried out intraocularly (intravitreally). Administration can be carried out ophthalmically. Administration can be carried out systemically. Administration may be carried out intranasally. Administration can be carried out intravenously. Administration can be carried out intraperitoneally. Administration can be carried out intradermally. Administration can be carried out intrathecally. Administration can be carried out intracerebroventricularly. Administration can be carried out iontophoretically. Administration can be carried out transmucosally. Administration can be carried out intravitreally. Administration can be carried out intramuscularly. Administration includes self-administration, administration by another or administration by a device (e.g., a pump).

As used herein, to “ameliorate” or “ameliorating” a disease, disorder or condition refers to results that, in a statistical sample or specific subject, make the occurrence of the disease, disorder or condition (or a sign or symptom thereof) better or more tolerable in a sample or subject administered a therapeutic agent or agents relative to a control sample, control subject or control group of subjects.

As used herein, the term “amino acid” includes naturally-occurring amino acids and synthetic amino acids, as well as amino acid analogs and amino acid mimetics that function in a manner similar to the naturally-occurring amino acids. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH. Naturally-occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, nor leucine, methionine sulfoxide, methionine methyl sulfonium. Such analogs have modified R groups (e.g., nor leucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “bevemipretide” refers to a peptidomimetic having a name: (R)-2-amino-N—((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide, having the chemical abstract service number CAS #: 2356106-71-1), and structure:

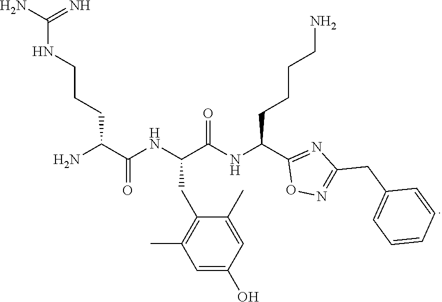 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

For simplicity of use herein, “bevemipretide” also refers to and includes all salts forms thereof (e.g., the tris-HCl salt of formula:

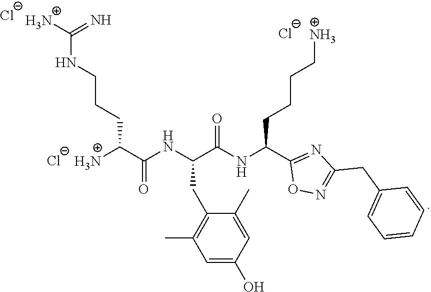 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

as well as all stereoisomers, tautomers, hydrates, solvates, formulations, and forms of all of the forgoing.

As used herein, the phrase “delaying (slowing) the progression of” refers to, in a statistical sample, slowing the rate at which a disease, disorder or condition, or the rate at which one or more signs or symptoms of the disease, disorder or condition progresses, in a sample or subject administered a therapeutic agent or agents relative to a control sample, control subject or control group of subjects. For example, in the current context, delaying (slowing) thinning of the ONL and/or reduction in total retinal thickness could be determined, for example, by showing that the treated subject or treated group of subjects exhibit slower progression of ONL thinning or reduction in total retinal thickness over time as compared with an untreated subject or untreated control group of subjects.

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition, or the physiological signs or symptoms of the disease, disorder or condition The amount of a composition administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent(s) could be administered simultaneously, sequentially or by separate administration). The present technology relates to the administration of bevemipretide, optionally with co-administration of one or more additional therapeutic agents.

As used herein, the term “hydrate” refers to a compound which is associated (e.g., complexed) with water. The number of the water molecules contained in a hydrate of a compound may be (or may not be) in a definite ratio to the number of the compound molecules in the hydrate.

As used herein, “inhibit” or “inhibiting” refers to the reduction in a sign or symptom (e.g. risk factor) associated with a disease, disorder or condition by an objectively measurable amount or degree compared to a control. In one embodiment, inhibit or inhibiting refers to the reduction by at least a statistically significant amount compared to a control sample, control subject or group of control subjects. In one embodiment, inhibit or inhibiting refers to a reduction by at least 5 percent compared to control sample, control subject or group of control subjects. In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control sample, control subject or group of control subjects.

As used herein “optical coherence tomography” or “OCT” refers to a noninvasive imaging modality using a beam of light (i.e., light interference) to rapidly scan the eye to generate direct cross-sectional image of the subject's retina and optic nerve suitable to produce measurements of the thickness/volume of individual layers/structures of the subject's eye. There are various types of OCT that can be used to produce a suitable OCT scan for the practice of this disclosure, including, but not limited to, time-domain OCT (i.e., TD-OCT), spectral domain OCT (i.e., SD-OCT) and swept-source (SS-OCT). If not otherwise specified, when used herein, a general reference to “optical coherence tomography” or “OCT” refers to all of the above forms of optical coherence tomography and any other form of optical coherence tomography now available or later developed and used for the examination a subject's/patient's eye(s).

As used herein “outer nuclear layer” or “ONL” refers to the retinal layer of the eye that contains the cell bodies of rod and cone photoreceptors.

The terms “pharmaceutically acceptable carrier” and “carrier” as used herein refer to a diluent, adjuvant, excipient, or vehicle with which a compound/drug product/composition (including a formulation or medicament) is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, “prevention” or “preventing” of a disease, disorder, or condition refers to results that, in a statistical sample, exhibit a reduction in the occurrence of the disease, disorder, or condition in a sample or subject administered a therapeutic agent or agents relative to a control sample, control subject, or group of control subjects or otherwise causes a delay in the onset of one or more symptoms of the disease, disorder, or condition relative to the control sample, control subject or group of control subjects. Such prevention is sometimes referred to as a prophylactic treatment.

As used herein “protecting” refers to keeping a tissue, cell, organ or organ substructure from damage, harm or injury or otherwise lessening the damage, harm or injury that would otherwise occur to the tissue, cell, organ or organ substructure in the absence of the administration of a protective agent (in this case administration of bevemipretide, or a pharmaceutically acceptable salt, stereoisomer, tautomer, hydrate, and/or solvate thereof).

In the context of therapeutic use or administration, the term “separate” or “separately” refers to an administration of at least two active pharmaceutical ingredients (i.e., therapeutic agents) by different routes, formulations, and/or pharmaceutical compositions.

As used herein, the term “sequential” therapeutic use refers to administration of at least two active pharmaceutical ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the term “solvate” refers to forms of a compound (e.g., peptide, peptidomimetic, aptamer or conjugate) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, the terms “subject” and “patient” are used interchangeably and each refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

It is also to be appreciated that the various modes of treatment or prevention of medical conditions as described herein, in some embodiments, are intended to mean “substantial,” which includes total but also less than total treatment or prevention, and wherein some biologically or medically relevant result is achieved.

As used herein, a “synergistic therapeutic effect” refers to a greater-than-additive therapeutic effect which is produced by a combination of at least two agents, and which exceeds that which would otherwise result from the individual administration of the agents.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “treating” or “treatment” refer to therapeutic treatment, wherein the object is to reduce, alleviate or slow down (lessen) a pre-existing disease, disorder, or condition or its related signs, or symptoms. By way of example, but not by way of limitation, a subject is successfully “treated” for a disease if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, or symptoms or conditions associated with the disease, disorder or condition relative to a control sample, control subject or control group of subjects.

### Therapeutic and Prophylactic Methods:

In some embodiments, bevemipretide, or a pharmaceutically acceptable salt, stereoisomer, tautomer, hydrate, and/or solvate thereof, is administered to a subject in need thereof to prevent thinning of the retinal outer nuclear layer (ONL) and/or reduction in total retinal thickness. Thinning of the ONL and/or reduction in the total retinal thickness can be determined using optical coherence tomography (OCT). Signs and symptoms associated with thinning ONL and/or reduction in total retinal thickness include, but are not limited to, blurred or distorted vision, difficulty seeing in dim lighting, loss of peripheral vision, floating or flashing lights in the field of view, and/or reduced color perception.

In some embodiments, bevemipretide, or a pharmaceutically acceptable salt, stereoisomer, tautomer, hydrate, and/or solvate thereof, is administered to a subject in need thereof to protect the ONL and/or retina from light-, or age-induced damage. Light- or age-induced damage can be determined by the thinning of the ONL and/or reduction in the total retinal thickness; each of which can be determined using optical coherence tomography (OCT). In some embodiments, the thinning of the ONL and/or reduction in the total retinal thickness results from light- or age-induced damage to photoreceptors.

In some embodiments, bevemipretide, or a pharmaceutically acceptable salt, stereoisomer, tautomer, hydrate, and/or solvate thereof, is administered to a subject in need thereof to protect photoreceptors from light- or age-induced damage. Light-, or age-induced damage of photoreceptors can be determined by the thinning of the ONL and/or reduction in the total retinal thickness; each of which can be determined using optical coherence tomography (OCT). In some embodiments, administration of bevemipretide, or a pharmaceutically acceptable salt, stereoisomer, tautomer, hydrate, and/or solvate thereof, protects the photoreceptors from age-induced damage. In some embodiments, administration of bevemipretide, or a pharmaceutically acceptable salt, stereoisomer, tautomer, hydrate, and/or solvate thereof, protects the photoreceptors from light-induced damage.

### Determination of the Biological Effect of Therapy:

In various embodiments, suitable in vitro or in vivo assays can be performed to determine the effect of a specific therapeutic and prophylactic methods described herein. In various embodiments, in vitro assays can be performed with representative cells of the type(s) involved in the subject's disorder, to determine if a given method exerts the desired effect upon the cell type(s). Compounds for use in therapy or prevention can be tested in suitable animal model systems. Similarly, for in vivo testing, any of the animal model system known in the art can be used prior to administration to human subjects. In some embodiments, testing can be carried out on organelles and organoids created specifically for drug testing applications.

### Animal Models:

Compounds for use in therapy can be tested in suitable animal model systems including, but not limited to, rats, mice, chicken, cows, monkeys, rabbits, and the like, prior to testing in human subjects. Similarly, for in vivo testing, any of the animal model systems known in the art can be used prior to administration to human subjects.

### Modes of Administration, Compositions, Formulations, Medicaments, and Effective Dosages:

Any method known to those in the art for contacting a cell, organ or tissue with bevemipretide, or pharmaceutically acceptable salts, stereoisomers, tautomers, hydrates, and/or solvates thereof may be used. In some embodiments, the cell, organ or tissue is contacted with bevemipretide, or pharmaceutically acceptable salts, stereoisomers, tautomers, hydrates, and/or solvates thereof. Suitable methods include in vitro, ex vivo, or in vivo methods. In vivo methods typically include the administration of bevemipretide, or pharmaceutically acceptable salts, stereoisomers, tautomers, hydrates, and/or solvates thereof to a mammal, such as a human. When used in vivo for therapy, bevemipretide, or pharmaceutically acceptable salts, stereoisomers, tautomers, hydrates, and/or solvates thereof, can be used. The dose and dosage regimen will depend upon the degree of the disease, disorder or condition in the subject, the characteristics of the particular bevemipretide, or pharmaceutically acceptable salts, stereoisomers, tautomers, hydrates, and/or solvates thereof used, e.g., its therapeutic index, the subject, and the subject's history.

The effective amount may be determined during pre-clinical trials and clinical trials by methods familiar to physicians and clinicians. An effective amount of bevemipretide, or pharmaceutically acceptable salts, stereoisomers, tautomers, hydrates, and/or solvates thereof useful in the methods may be administered to a mammal in need thereof by any of a number of well-known methods for administering pharmaceutical compounds. For example, the peptidomimetic may be administered subcutaneously, topically, intraocularly (intravitreally), ophthalmically, orally, intranasally, systemically, intravenously, intraperitoneally, intradermally, intrathecally, intracerebroventricularly, iontophoretically, transmucosally, or intramuscularly.

The bevemipretide, or its stereoisomers, tautomers, hydrates, and/or solvates thereof may be formulated from a pharmaceutically acceptable salt.

Bevemipretide, or pharmaceutically acceptable salts, stereoisomers, tautomers, hydrates, and/or solvates thereof can be incorporated into pharmaceutical compositions (e.g., a formulation or medicament) for administration, singly or in combination, to a subject for the treatment or prevention of a disease, disorder or condition described herein. Bevemipretide, or pharmaceutically acceptable salts, stereoisomers, tautomers, hydrates, and/or solvates thereof may be formulated with other compounds such as a therapeutic agent, a peptide, another peptidomimetic, or mixtures thereof. Such pharmaceutical compositions typically include the active agent and a pharmaceutically acceptable carrier. In some embodiments, the pharmaceutical compositions can be used as medicaments or in the preparation of medicaments for administration to a subject suffering from or at risk for thinning ONL and/or reduction in total retinal thickness. In some embodiments, the pharmaceutical compositions can be used as medicaments or in the preparation of medicaments for administration to a subject suffering from light- or age-induced damage of the ONL or retina, and/or (ii) light- or age-induced damage of photoreceptors. Pharmaceutically acceptable carriers include saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions (e.g., a formulation or medicament) can be formulated to be compatible with its intended route of administration. Examples of routes of administration of the pharmaceutical composition include parenteral (e.g., intravenous, intradermal, intraperitoneal or subcutaneous), oral, systemic, inhalation, transdermal (topical), intraocular (intravitreal), ophthalmic, intrathecal, intracerebroventricular, iontophoretic, transmucosal, intravitreal and intramuscular administration. In some embodiments, the route of administration of the pharmaceutical composition is oral. In some embodiments, the route of administration of the pharmaceutical composition is subcutaneous. In some embodiments, the route of administration of the pharmaceutical composition is topical. In some embodiments, the route of administration of the pharmaceutical composition is intraocular. In some embodiments, the route of administration of the pharmaceutical composition is ophthalmic. Other suitable methods are discussed elsewhere herein.

Solutions or suspensions (e.g., a formulation or medicament) used for parenteral, intradermal, subcutaneous, intraocular (intravitreal), or ophthalmic application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a dosing/treatment course (e.g., 7 days or more of administration or 30 days or more of administration).

Pharmaceutical compositions (e.g., a formulation or medicament) suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). A composition for administration by injection will generally be sterile and should be fluid to the extent that easy syringability exists. It should be stable under the conditions of manufacture and storage and should be preserved against the contaminating action of microorganisms such as bacteria and fungi.

The bevemipretide (or pharmaceutically acceptable salts, stereoisomers, tautomers, hydrates, and/or solvates thereof)-containing compositions (e.g., a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions (e.g., a formulation or medicament) can be prepared by incorporating the active compound (e.g., bevemipretide) in the required amount in an appropriate solvent with one or a combination of ingredients enumerated above, as required, followed by filtered sterilization. Generally, dispersions are prepared by incorporating the active compound (e.g., bevemipretide) into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions (e.g., formulations or medicaments) generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

One may dilute or increase the volume of a composition, formulation or medicament comprising a compound, therapeutic agent, peptide, peptidomimetic or mixtures thereof with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the composition, formulation or medicament comprising compound, therapeutic agent, peptide, peptidomimetic or mixtures thereof with an inert material into a solid dosage form. Materials used as disintegrates include but are not limited to starch, including the commercial disintegrant based on starch, e.g., Explotab®. Sodium starch glycolate, Amberlite®, sodium carboxymethylcellulose, ultramylopectin, sodium alginate, gelatin, orange peel, acid carboxymethyl cellulose, natural sponge and bentonite may all be used as disintegrants. Another form of the disintegrants are the insoluble cationic exchange resins. Powdered gums may be used as disintegrants and as binders, and these can include powdered gums such as agar, karaya or tragacanth. Alginic acid and its sodium salt are also useful as disintegrants.

Binders may be used to hold a compound, therapeutic agent, peptide, peptidomimetic or mixtures thereof in a formulation with an inert material together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the formulation.

An anti-frictional agent may be included in the composition, formulation or medicament comprising a compound, therapeutic agent, peptide, peptidomimetic or mixtures thereof to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of a compound, therapeutic agent, peptide, peptidomimetic or mixtures thereof into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or medicament comprising a compound, therapeutic agent, peptide, peptidomimetic or mixtures thereof of the technology or derivative either alone or as a mixture in different ratios.

Pharmaceutical compositions (e.g., a formulation or medicament) which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All compositions, formulations, or medicaments for oral administration should be in dosages suitable for such administration.

For administration of a composition, formulation, medicament or compound by inhalation for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the composition, formulation, medicament, or compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

A compound, composition (e.g., formulation or medicament), therapeutic agent, peptide, peptidomimetic or mixtures thereof can be delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995, to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

For ophthalmic or intraocular (intravitreal) formulations, any suitable mode of delivering bevemipretide, or pharmaceutically acceptable salts, stereoisomers, tautomers, hydrates, and/or solvates thereof (with or without therapeutic agents, peptides or other peptidomimetics), to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of formulations suitable for administration in or near the eye include, but are not limited to, ocular inserts, minitablets, and topical formulations such as eye drops, ointments, and in situ gels. In one embodiment, a contact lens is coated with bevemipretide, or pharmaceutically acceptable salts, stereoisomers, tautomers, hydrates, and/or solvates thereof. In some embodiments, a single dose comprises from between 0.1 ng to 5000 μg, 1 ng to 1000 μg, or 10 ng to 200 μg of bevemipretide, or pharmaceutically acceptable salts, stereoisomers, tautomers, hydrates, and/or solvates thereof administered to the eye.

Eye drops can comprise a sterile liquid formulation that can be administered directly to the eye. In some embodiments, eye drops comprising bevemipretide, or pharmaceutically acceptable salts, stereoisomers, tautomers, hydrates, and/or solvates thereof can be used and may further comprise one or more preservatives. In some embodiments, the optimum pH for eye drops comprising bevemipretide is about 5.6-6.0, inclusive. In some embodiments, the pH is 5.7, 5.8, or 5.9. In some embodiments, the pH is 5.8. In some embodiments, the bevemipretide, or pharmaceutically acceptable salts, stereoisomers, tautomers, hydrates, and/or solvates thereof is administered via eye drops, wherein the eye drops comprise the bevemipretide, or pharmaceutically acceptable salts, stereoisomers, tautomers, hydrates, and/or solvates thereof in a concentration of between 0.2% and 5%, inclusive (wt./vol.). In some embodiments, the eye drops further comprise water, a buffer, and/or a preservative. In some embodiments, the preservative is benzalkonium chloride (BAK). In some embodiments, the eye drops are administered to an eye once daily or multiple times (e.g., twice, three times or more) per day in a volume of about 5 μL to about 50 μL per dose, per eye.

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, silicon containing formulations and cellulose acetate phthalate. In some embodiments, the compound, therapeutic agent, peptide, peptidomimetic or mixtures thereof is formulated into an in-situ gel (as the pharmaceutical composition).

For topical ophthalmic administration, a compound, therapeutic agent, peptide, peptidomimetic or mixtures thereof may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more peptidomimetics, slow release of one or more peptidomimetics with constant speed and limiting of one or more peptidomimetics' systemic absorption. In some embodiments, an ocular insert comprises bevemipretide, or pharmaceutically acceptable salts, stereoisomers, tautomers, hydrates, and/or solvates thereof and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers.

Minitablets are biodegradable, solid drug forms, that transit into gels after application to the conjunctival sac, thereby extending the period of contact between active ingredient and the eyeball surface, which in turn increases the active ingredient's bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise bevemipretide, or pharmaceutically acceptable salts, stereoisomers, tautomers, hydrates, and/or solvates thereof and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol or Carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular (intravitreal) preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising bevemipretide, or pharmaceutically acceptable salts, stereoisomers, tautomers, hydrates, and/or solvates thereof is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

Systemic administration of a compound, composition (e.g., formulation or medicament), therapeutic agent, peptide, peptidomimetic or mixtures thereof, as described herein, can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A compound, composition (e.g., formulation or medicament), therapeutic agent, peptide, peptidomimetic or mixtures thereof can be formulated in a carrier system. The carrier can be a colloidal system. The colloidal system can be a liposome, a phospholipid bilayer vehicle. In one embodiment, the compound, composition (e.g., formulation), therapeutic agent, peptide, peptidomimetic or mixtures thereof is encapsulated in a liposome while maintaining integrity of the compound, therapeutic agent, peptide, peptidomimetic or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the compound, composition (e.g., formulation), therapeutic agent, peptide, peptidomimetic or mixtures thereof can be embedded in the polymer matrix, while maintaining integrity of the composition. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the compound, composition (e.g., formulation), therapeutic agent, peptide, peptidomimetic or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such compositions/formulations/medicaments can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds (e.g., bevemipretide) can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro.

In addition to the formulations described above, compound, compositions, therapeutic agent, peptide, peptidomimetic or mixtures thereof may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

A compound, composition, therapeutic agent, peptide, peptidomimetic or mixtures thereof may be provided in particles or polymer microspheres. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The compounds, compositions, therapeutic agents, peptides, peptidomimetics or mixtures thereof also may be dispersed throughout the particles. The compounds, compositions, therapeutic agents, peptides, peptidomimetics or mixtures thereof also may be adsorbed into the particles. The particles may be of any order release kinetics, including zero-order release, first-order release, second-order release, delayed release, sustained release, immediate release, and any combination thereof, etc. The particle may include, in addition to the compounds, compositions, therapeutic agents, peptides, peptidomimetics or mixtures thereof, any of those materials routinely used in the art of pharmacy and medicine, including, but not limited to, erodible, nonerodable, biodegradable, or nonbiodegradable material or combinations thereof. The particles may be microcapsules which contain the compound of the technology in a solution or in a semi-solid state. The particles may be of virtually any shape.

Both non-biodegradable and biodegradable polymeric materials can be used in the manufacture of particles for delivering the compounds, compositions, therapeutic agents, peptides, peptidomimetics or mixtures thereof. Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and polycaprolactone.

The compounds, compositions, therapeutic agents, peptides, peptidomimetics or mixtures thereof may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant (depot) is constructed and arranged to deliver therapeutic levels of the active ingredient (i.e. compound, therapeutic agent, peptide, peptidomimetic or mixtures thereof) for at least 7 days, for at least 30 days, for at least 60 days, for at least 90 days, for at least 120 days, for at least 180 days or for at least 365 days. In some embodiments, the “long-term” release means 30-60 days, 60-90 days, 90-120 days, 120-180 days, or 180-365 days. Long-term sustained release implants are well-known to those of ordinary skill in the art and include some of the release systems described above.

Dosage, toxicity and therapeutic efficacy of any compounds, compositions (e.g., formulations), therapeutic agents, peptides, peptidomimetics or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

In some embodiments, an effective systemic dose of the peptidomimetics, sufficient for achieving a therapeutic or prophylactic effect, range from about 0.002 mg per kilogram body weight per day to about 2 mg per kilogram body weight per day. Suitably, the dosage ranges are from about 0.01 mg per kilogram body weight per day to about 0.5 mg per kilogram body weight per day. For example dosages can be 0.03-0.3 mg/kg body weight every day or 0.05-0.5 mg/kg body weight every two days or every three days or within the range of 0.1-1 mg/kg every week, every two weeks or every three weeks. In one embodiment, a single dosage of peptide or peptidomimetic ranges from 10-1000 micrograms per kg body weight. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

In some embodiments, a therapeutically effective amount of a peptidomimetic may be defined as a concentration of peptidomimetic at the target tissue of 10-9 to 10-5 molar, e.g., approximately 10-7 molar. This concentration may be delivered by systemic doses of 0.01 to 10 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application). Dosing using optical eye drop solutions are discussed elsewhere herein.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the compounds, therapeutic agents, peptides, peptidomimetics or mixtures thereof described herein can include a single treatment or a series of treatments.

### EXAMPLES

The present technology is further illustrated by the following examples, which should not be construed as limiting in any way.

### Example 1—Preservation of Photoreceptors, Outer Nuclear Layer (ONL) and Retina/Retinal Tissue with Bevemipretide

### Introduction

The retinal outer nuclear layer (ONL) contains the nuclei of the photoreceptor cells. Photoreceptor loss leads to thinning of the ONL and a reduction in total retinal thickness, resulting in progressive vision loss. This example demonstrates the efficacy of bevemipretide in methods for preserving layers of photoreceptor cell nuclei in the ONL and overall retinal thickness in a rodent model of bright light-induced retinal degeneration.

### Methods:

The objective of this study was to evaluate the retinoprotective effects of bevemipretide in a white light-induced retinal degeneration (LIRD) model in Brown Norway rats.

TABLE 1
Experimental Design

| | | | | | | | Time of |
|---|---|---|---|---|---|---|---|
| | | OU | | | | | Euthanasia |
| Group | | | | Volume/ROA/ | Light | In-Life | Tissue |
| # | N | Tx | Dose | Schedule | Induction | Assessments | Collection |
| 1 | 6 | Vehicle | N/A | 5 μL/Topical BID | Day 3 | OEs: | Day 17: |
| 2 | 6 | Bevemipretide | 2% | (Preload Dose: Days | 1 hour | Baseline, D8, | Terminal |
| | | 2% (wt./vol.)* | | 1-2; 1^{st} dose | 10,000 lux | D16 | Blood |
| | | | | Immediately prior to | | OCTs: | Collected |
| | | | | Induction on Day 3, | | Baseline, D9, | on all |
| | | | | 2^{nd} dose 6-8 hrs | | D17 | animals |
| | | | | later; Post Induction | | | n = 3 eyes |
| | | | | Doses: Days 4-16) | | | per group |
| | | | | | | | enucleated |
| | | | | | | | for IHC |
| | | | | | | | n = 1 |
| | | | | | | | eye/animal/group |
| | | | | | | | enucleated |
| | | | | | | | for H&E |
| | | | | | | | For all |
| | | | | | | | remaining |
| | | | | | | | eyes: |
| | | | | | | | Enucleate, |
| | | | | | | | dissect and |
| | | | | | | | snap freeze |
| | | | | | | | for |
| | | | | | | | potential |
| | | | | | | | PK analysis |

BID = 2 doses, 6-8 hours apart

TABLE 2
Test System: Animals, Housing, and Environmental Conditions

| | |
|---|---|
| Species/Strain | Rat (Rattus norvegicus)/Brown Norways |
| Source | Charles River |
| Age Range at First Dosing | Approximately 8-12 weeks |
| Weight Range at First Dosing | 200 -350 g |
| Identification | Tail marking and cage card |
| Physical examination Time | During acclimation |
| Caging | Innovive ® disposable rat caging |
| Environmental Conditions | Photoperiod: 12 hrs. light/12 hrs. darkness |
| | Temperature: 68-79° F. |

TABLE 3
Animal Diet and Water

| | | |
|---|---|---|
| Feed | Type | Lab Diet |
| | Name | Lab Diet 5002 |
| | Availability | ad libitum |
| | Analysis for contaminants | Not routinely performed; No contaminants |
| | | suspected |
| Water | Source | Durham City Water |
| | Availability | ad libitum via water bottles with sipper tubes |
| | Analysis for contaminants | Not routinely performed; No contaminants |
| | | suspected |

TABLE 4
Test Article Description

| | |
|---|---|
| Test Article (Group 1) | Vehicle (NaCl + NaH₂PO₄ + H₂O) |
| Concentration | NA |
| Storage Conditions | Refrigerated (2-8° C.) |
| Test Article (Group 2) | Bevemipretide (2% weight/volume (i.e., |
| | wt./vol.) topical solution)* |
| Concentration | 2%, 20 mg/mL* |
| Storage Conditions | Refrigerated (2-8° C.) |

*based on free-base weight of bevemipretide using bevemipretide tris-HCl to prepare the formulation and adjusting for potency

Animal health and acclimation. Animals were acclimated to the environment for a minimum of 3 days. At the completion of the acclimation period, each animal was physically examined for determination of suitability for study participation. Examination included the skin and external ears, eyes, and abdomen, neurological behavior, and general body condition. Animals determined to be in good health were released to the study.

Randomization and study identification. Animals were assigned to study groups according to standard operating procedures. Animals were uniquely identified by corresponding cage card number and tail marking.

Topical dosing (Groups 1 and 2). Topical application of vehicle or bevemipretide (2% (weight/volume) topical solution) was completed twice daily (BID) for Groups 1 and 2, respectively, during which time a volume of 5 μL vehicle or bevemipretide was administered to the ocular surface of both eyes (OU) via calibrated air displacement pipette. With the animal manually restrained, the upper eyelid of one eye was gently elevated to expose the cornea, and the bevemipretide was applied to the corneal surface, taking care to avoid contacting the eye with the pipette tip. The contralateral eye was dosed in an identical fashion. The animal was then allowed to blink several times while still manually strained to distribute the applied solution over the eye. If needed, blinking was induced with gentle tapping beneath the eye. The animal was then returned to its cage. The animals received their first dose on day 3 immediately prior to being placed in the induction chamber.

Light-induced retinal degeneration induction. Beginning the night before induction of retinal degeneration through bright light damage, animals were dark adapted. Prior to the procedure, animals received buprenorphine (0.03 mg/kg SQ) for pain management. A photometer was placed in the center of an empty, transparent rodent cage, and two white lights were placed on the long sides of the cage to measure 10,000 lux pointing directly at each light. A measurement was then taken from the center of the cage with the lux meter positioned vertically to get a combined reading of approximately 10,000 lux. Ten minutes prior to model induction, under dim red light, mydriasis was induced with 1 drop each of Tropi-Phen (1% tropicamide HCl, 10% phenylephrine hydrochloride) and 1% atropine, applied topically OU. Animals were then placed in the cage and exposed to the white light for one hour. Following light exposure, the animals were returned to their cage and a normal light/dark cycle was resumed.

Cage side clinical observations and morbidity monitoring. All animals underwent twice daily cage side clinical observations with particular attention paid to the eyes. All animals were assessed for morbidity twice daily, morning and afternoon.

Body weights. All animals were weighed prior to dosing, weekly, and at time of necropsy.

Ocular examinations (OF). Ocular examinations were performed using a slit lamp biomicroscope and indirect ophthalmoscope and lens to evaluate anterior and posterior segment clinical observations for all animals at the timepoints indicated (baseline, days 8 and 16). A topical mydriatic (Tropi-Phen (1% tropicamide and 2.5% phenylephrine HCl per eye)) was given following the anterior segment examination to facilitate examination of the posterior segment. Animals were not tranquilized for the examinations.

Optical coherence tomography (OCT)—Sedation. Animals were sedated with inhaled isoflurane in oxygen (0.5-3%). Alternatively, animals may be sedated with a ketamine/xylazine cocktail (60-80/7.5-10 mg/kg IP).

Optical coherence tomography (OCT)—Preparation and Procedure. At least fifteen minutes prior to examination, a cocktail of 1% tropicamide HCl and 2.5% phenylephrine hydrochloride was applied topically to dilate and proptose the eyes, if needed. The procedure was performed by a trained technician. Animals underwent OCT imaging procedures of the posterior section of the eye at baseline and on Days 9 and 17 to determine retinal changes over time using a Heidelberg Spectralis OCT. A series of b-scans were collected to capture the entire posterior segment with the optic nerve head (ONH) oriented centrally. The outer nuclear layer (ONL) thickness and total retinal thickness (TRT) were measured at three positions (left, right, and center) from the superior OCT scan. Following OCT procedures, animals were returned to their cages and allowed to recover normally or were euthanized as indicated in the experimental design table.

Optical coherence tomography (OCT)—Post-Procedural Care. Following the procedure, eye lubrication was applied topically to the ocular surface. If needed, animals received atipamezole (0.1-1.0 mg/kg (1 mg/mL), intramuscularly (IM) or intraperitoneally (IP)) to reverse the effects of xylazine. Animals were returned to their cages and allowed to recover normally. No special post-procedural care was necessary aside from assuring the animals resumed normal activity. Alternatively, animals were euthanized following the final collection interval.

Optical coherence tomography (OCT)—Data Analytics. Outer nuclear layer (ONL) and total retinal layer thickness was measured at three positions (left, right, and center) from a superior OCT scan.

Euthanasia Tissue Collections. At the timepoints indicated in the experimental design, animals were euthanized via carbon dioxide asphyxiation and full blood volume was collected from all study animals at necropsy. Following euthanasia, all eyes were enucleated and processed.

Terminal blood collection for plasma. Full blood volume was collected from all study animals at necropsy. Immediately following euthanasia with CO₂, a 25G needle was inserted into the heart and the animal was exsanguinated and death was confirmed by cervical dislocation or thoracotomy (if >200 g). Whole blood was deposited into K2EDTA tubes for plasma acquisition. After collection, the tubes were gently mixed by inverting the tubes 5-8 times. Blood samples were stored on wet ice for up to 30 minutes prior to plasma processing. The samples were centrifuged at 4° C. for 10 minutes at 2000 g in a swinging bucket refrigerated centrifuge. Immediately after centrifugation, the clear plasma was transferred to a prelabelled 2 mL screw-cap polypropylene tube and stored frozen at −80° C.

Eyes Designated for Immunohistochemistry (IHC). Immediately following euthanasia and confirmation of death, all eyes designated for IHC were enucleated and marked superiorly (silver sharpie) at the 12 o'clock position. A slit across the front of the cornea from limbus to limbus was performed and then each globe was placed into 4% paraformaldehyde (PFA) in phosphate-buffered saline (PBS). Eyes were fixed at 4° C. overnight. Following fixation, eyes were transferred into PBS, brought through a sequential sucrose gradient (10-30%, 1 hour each), followed by a 20-minute incubation in 50:50 30% sucrose and OCT at room temperature. Eyes were then embedded in OCT medium and stored at −80° C. for sectioning and staining. Upon election for IHC, blocks from Groups 1 and 2 were section (14 μm sections), picking up 10 central slides (8 sections/slide). Two central slides per eye were selected for immunofluorescence (IF) staining. Slides were rehydrated in PBS and then permeabilized in PBS containing 0.1% Triton X-100 for 2 hours at room temperature. During the equilibration step, the TdT solution for each slide was prepared as follows: 135 μL equilibration buffer, 15 μL nucleotide mix, and 3 μL rTdT enzyme. TdT reaction mixture (150 μL) was added to each slide, and the slides were incubated covered at 37° C. for 1 hour. The reaction was terminated by adding 200 μL/slide of 2×SSC (20×SSC diluted 1:10 in dH2O) and incubating for 15 min at room temperature (RT). Slides were washed 3×5 minutes in PBS at RT and then incubated with primary antibodies overnight at 4° C. as shown in Table A.

TABLE A
Primary and Secondary Antibody Diluent: PBS containing 2% normal
donkey serum (NDS) and 2% bovine serum albumin (BSA).

| Primary Antibodies | Secondary Antibodies |
|---|---|
| (o/n @ 4° C.) | (RT × 2 h protected from light) |
| 1/200 rabbit anti-RPE65 | 1/200 goat anti-rabbit AlexaFluor750 |
| (Abcam, cat# ab231782) | (Thermo, cat# A-21039) |
| 1/250 mouse anti-Rhodopsin | 1/200 donkey anti-mouse Cy3 |
| (Abcam, cat# ab5417) | (Jackson Immuno, cat# 715-165-151) |
| — | |
| — | 1/1000 DAPI |

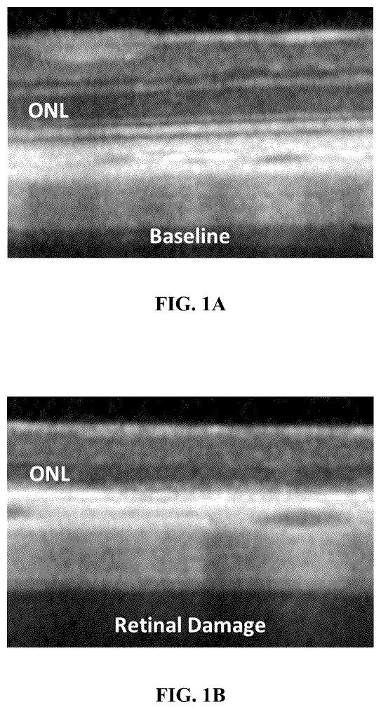

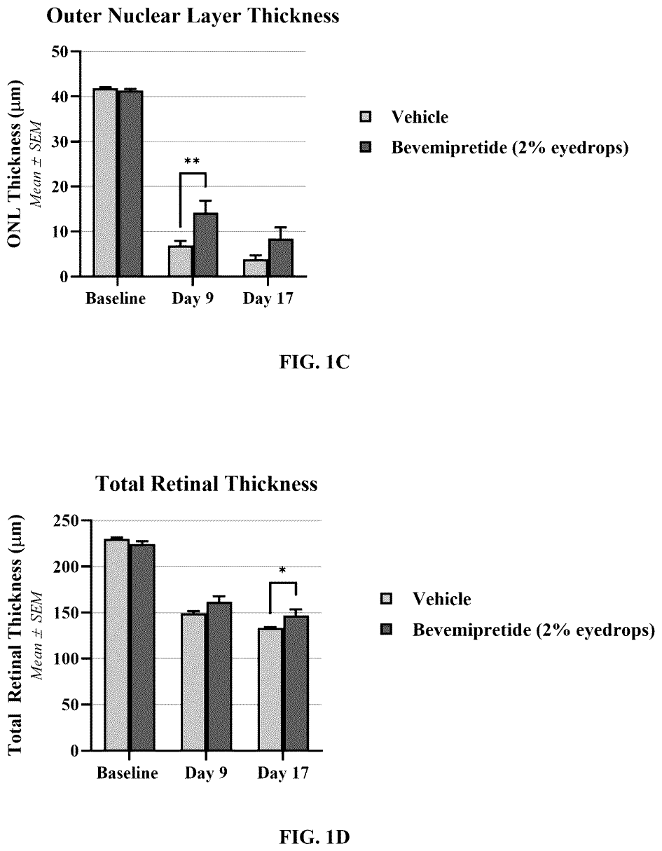

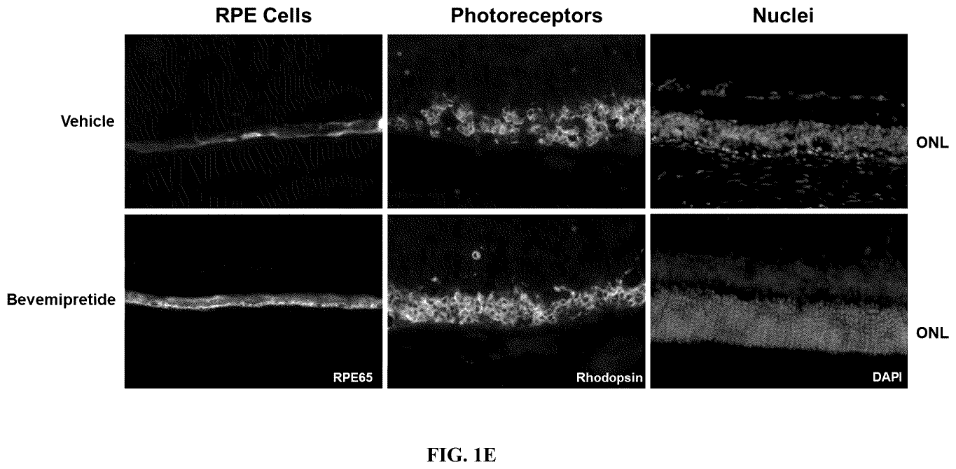

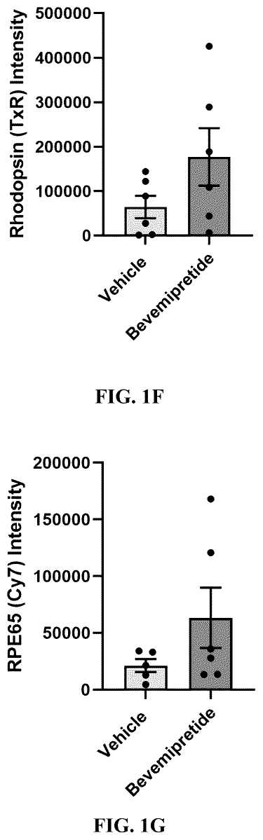

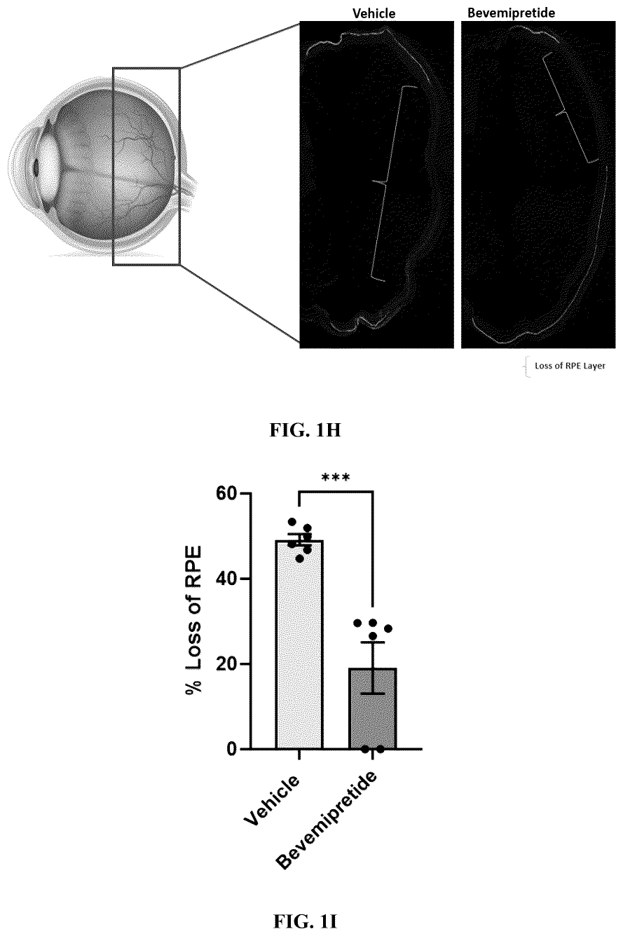

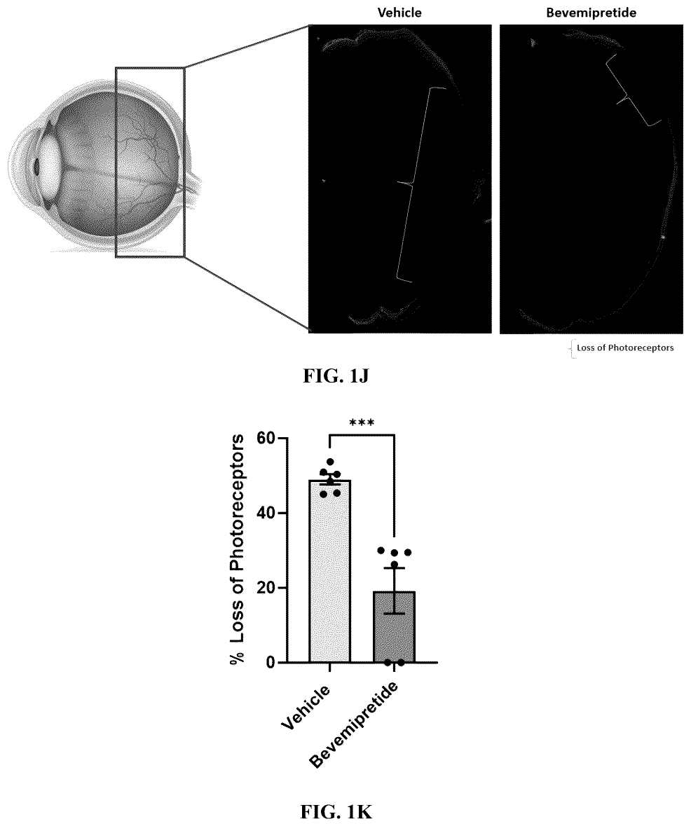

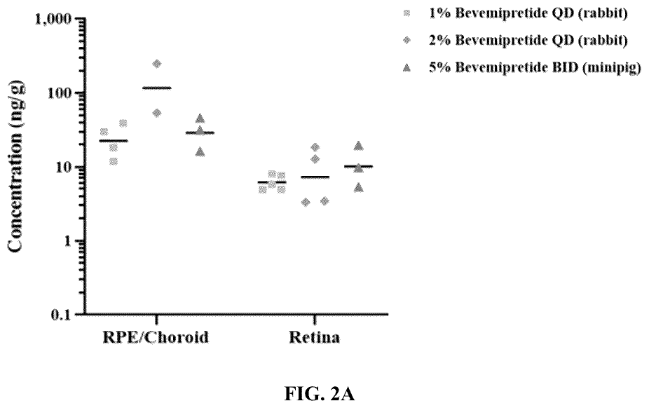

Following overnight incubation and 3×5 minute washes in PBS, slides were incubated in the secondary antibody cocktail listed above for 2 hours at room temperature. Following 3 final washes, slides were mounted and cover-slipped. Slides were screened for retinal pigment epithelium (RPE) cell and photoreceptor integrity. Sections with a representative portrayal of retinal architecture were selected for imaging. A 4× overview and a 20× high magnification image were acquired using a fluorescent widefield microscope equipped with plan apochromatic objectives. Results are shown in FIG. 1E, FIG. 1H, and FIG. 1J. Analysis of the images in FIG. 1E are presented graphically in FIGS. 1F and 1G. Analysis of the images in FIG. 1H and FIG. 1J are presented graphically in FIGS. 1I and 1K, respectively.

### Results:

Preservation of ONI, and Total Retinal Thickness. Bright light exposure induced photoreceptor loss, as evidenced by the thinning of the ONL and reduction in total retinal thickness shown in OCT imaging after light exposure (FIGS. 1A-1D). Topical administration of bevemipretide (2% twice daily) starting 2 days before light exposure preserved ONL thickness and total retinal thickness relative to vehicle control (FIGS. 1C-1D).

As shown in FIG. 1E, the overall structure of retinal layers in bevemipretide-treated eyes appeared to retain a higher degree of organization, with increased staining for RPE cells and photoreceptors, and a more highly organized nuclear layer compared with vehicle-treated eyes. Staining analysis showed a trend for increased Rhodopsin (TxR; FIG. 1F) and RPE65 (Cy7; FIG. 1G) in bevemipretide-treated eyes as compared to vehicle-treated eyes.

As shown in FIGS. 1H-1I, vehicle-treated animals had larger regions of RPE loss compared to bevemipretide-treated animals, and 1 animal in the bevemipretide-treated group had disorganized retinal layers with no RPE loss (one animal, two images quantified). When the % loss of RPE cells was calculated, vehicle-treated eyes had a significantly higher loss of RPE cells compared to bevemipretide-treated eyes (p=0.0007, FIG. 1I). In addition, as shown in FIGS. 1J and 1K, animals had a significantly larger % loss of photoreceptors in vehicle-treated as compared to bevemipretide-treated eyes (p=0.0008, FIG. 1K).

### Conclusion:

Accordingly, these results demonstrate that the administration of topical bevemipretide is effective in methods for: (i) preventing the thinning of the outer nuclear layer (ONL); (ii) for preventing a reduction in total retinal thickness; (iii) protecting the retinal ONL and/or the retina from light- or age-induced damage; and/or (iv) protecting photoreceptors from light- or age-induced damage.

### Example 2—Delivery of Bevemipretide to Retinal Tissues with Low Systemic Exposure

### Methods:

The objective of this study was to evaluate the ocular tolerability and distribution of bevemipretide in New Zealand white rabbits following 29 days of topical dosing and Yucatan minipigs following 28 days of topical dosing.

TABLE 5
Experimental Design - New Zealand White Rabbits

| | | OU | | | |
|---|---|---|---|---|---|
| | | | RoA / | | |
| Group | | | Volume / | | Euthanasia** / |
| # | N | Tx | Frequency* | In-Life Assessments | Tissue Collections |
| 1 | 14 | Bevemipretide | Topical / | Ocular | N = 3 (OU) at Days |
| | | 1% (wt./vol.)^{&} | 35 μL / SID | Examinations: | 2, 15, 22, & 30 for |
| | | | | Baseline and prior to | plasma & frozen |
| | | | | first dose of day on | ocular dissections |
| 2 | 6 | Bevemipretide | | Days 2, 8, 15, 28 | N = 2 (OU) at Day |
| | | 2% (wt./vol.)^{&} | | | 30 for plasma and |
| | | | | | frozen ocular |
| | | | | | dissections |

Bevemipretide formulations were in 4.16 mg/mL sodium phosphate buffer; pH 5.8 (pH range 5.7-5.9). For isotonicity, the 1% formulation included 5.3 mg/mL NaCl, and the 2% formulation included 3.4 mg/mL NaCl.
*SID = approximately every 24 hours ± 2 hours (except on Day 2 due to time of TA arrival);
**Terminal timepoints for plasma collection and enucleation for ocular dissections performed at approximately 24 hours post-final dose.

TABLE 6
Test System: Animals, Housing, and Environmental
Conditions - New Zealand White Rabbits

| | |
|---|---|
| Species/Strain | Rabbit (Oryctolagus |
| | cuniculus)/New Zealand White |
| Source | Envigo, Denver, PA |
| Age Range at First Dosing | Approximately 4-6 months |
| Weight Range at First Dosing | 2.0-3.7 kg |
| Identification | Cage card and ear tag |
| Physical examination Time | During acclimation |
| Caging | Stainless steel; 17 inches wide × |
| | 7 inches deep × 15 inches tall |
| | or larger, slatted bottoms. No |
| | additional bedding. |
| Environmental Conditions | Photoperiod: 12 hrs. light/12 |
| | hrs. darkness |
| | Temperature: 68 ± 2° F. |

TABLE 7
Animal Diet and Water - New Zealand White Rabbits Feed

| | |
|---|---|
| Type | Hi Fiber Rabbit Diet |
| Name | Hi Fiber Lab Rabbit Diet #5P25, Purina, St. Louis, |
| | MO |
| Availability | ad libitum |
| Analysis for | Not routinely performed; No contaminants |
| contaminants | suspected |
| Source | Durham City Water |
| Availability | ad libitum via water bottles with sipper tubes |
| Analysis for | Not routinely performed; No contaminants |
| contaminants | suspected |

TABLE
Design - Yucatan Minipigs

| | | | ROA / | | |
|---|---|---|---|---|---|
| Group | | | Volume / | In-Life | Euthanasia / Tissue |
| # | N | Tx | Frequency | Assessments | Collections |
| 3 | 3 | Bevemipretide | Topical | Ocular | Day 29: |
| | | 5% (wt./vol.)^{&} | (OU) / | Examinations: | Blood collected for |
| | | | 50 μL / BID | Baseline and Days 3, | plasma |
| | | | | 7, 15, & 29 | Tissues dissected |
| | | | | Plasma | from OD eyes: |
| | | | | Collection %: 0.25 h, | Bulbar conjunctiva, |
| | | | | 0.5 h, 2 h, 4, 6 h, and | sclera, AH, lens, |
| | | | | 24h following first | VH, cornea, ICB, |
| | | | | dose on Days 1 and | retina, RPE/choroid |
| | | | | 28 | |

Bevemipretide formulation was in 4.16 mg/mL sodium phosphate buffer, pH 5.8 (pH range 5.7-5.9), with no added NaCl.
^{%}Second topical dose of day applied following 6 h blood collection.

TABLE 9
Test System: Animals, Housing, and Environmental
Conditions - Yucatan Minipigs

| | |
|---|---|
| Species/Strain | Swine/Yucatan Minipig |
| Source | Premier BioSource |
| Age Range at First Dosing | 2-4 months |
| Weight Range at First Dosing | 10-25 kg |
| Identification | Ear tag |
| Physical examination Time | During acclimation |
| Caging | Steel panels 15′ × 5′ with |
| | shavings on the floor, or raised |
| | flooring |
| Number per cage | Group-housed |
| Environmental Conditions | Photoperiod: 12 hrs light/12 hrs |
| | darkness |
| | Temperature: 68 ± 2° F. |

TABLE 10
Animal Diet and Water - New Zealand White Rabbits

| | | |
|---|---|---|
| Feed | Type | LabDiet |
| | Name | PMI 5084 |
| | Availability | Approximately ⅓-1 scoop twice a day |
| | Analysis for contaminants | Not routinely performed; No contaminants |
| | | suspected |
| Water | Source | Durham City Water |
| | Availability | ad libitum via water bowls with sipper |
| | | tubes/lixits |
| | Analysis for contaminants | Every 6 months, no contaminants found |

^{&} based on free-base weight of bevemipretide using bevemipretide tris-HCl to prepare the formulation and adjusting for potency

Animal Health and Acclimation. Animals were acclimated to the study environment for a minimum of 1 week prior to anesthesia or dosing. During or at completion of acclimation period, each animal was physically examined by a lab technician for suitability for study participation. Examination included the skin and external ears, eyes, abdomen, neurological behavior, and general body condition. Animals determined to be in good health were released to the study.

Mortality Morbidity. Animals were assessed for mortality or morbidity twice daily, morning and afternoon.

Cage-Side Clinical Observations. Animals underwent twice daily cage-side clinical observations with particular attention paid to the eyes and surrounding tissue for signs of irritation or inflammation.

Body weights. Animals were weighed prior to dosing, weekly, and at time of necropsy.

Topical Dosing—New Zealand White Rabbits (Groups 1 & 2). Topical application of bevemipretide was performed once daily, during which time a volume of 35 μL of bevemipretide topical solution (1% wt./vol. bevemipretide for Group 1 animals and 2% wt./vol. bevemipretide for Group 2 animals) was administered to the ocular surface of both eyes via calibrated air displacement pipette. With the animal manually restrained, the upper eyelid of one eye was gently elevated to expose the cornea, and bevemipretide topical solution was applied to the corneal surface, taking care to avoid contacting the eye with the pipette tip. The contralateral eye was dosed in an identical fashion. The animal was allowed to blink several times while still manually restrained to distribute the applied solution over the eye. If needed, blinking was induced with gentle tapping beneath the eye. The animal was then returned to its cage.

Topical Dosing—Yucatan Minipigs (Group 3). Topical application of bevemipretide topical solution (5% wt./vol. bevemipretide) was performed twice daily, during which time a volume of 50 μL of bevemipretide topical solution was administered to the ocular surface of both eyes via calibrated air displacement pipette. With the animal manually restrained, the upper eyelid of one eye was gently elevated to expose the cornea, and bevemipretide was applied to the corneal surface, taking care to avoid contacting the eye with the pipette tip. The contralateral eye was dosed in an identical fashion. The animal was allowed to blink several times while still manually restrained to distribute the applied solution over the eye. If needed, blinking was induced with gentle tapping beneath the eye. The animal was then returned to its pen.

Euthanasia. New Zealand white rabbits were sedated with ketamine/xylazine (20-60/4-15 mg/kg IM), blood was collected IC or IV, and euthanasia was performed with an overdose of sodium pentobarbital administered IC or IV, followed by auscultation or palpation to ensure death. Yucatan minipigs were sedated with ketamine (10 mg/kg) and dexmedetomidine (0.05 mg/kg) IM, then at least 3 mL of blood was collected via the intracardiac or intravenous route, and the animals were subsequently euthanized with an overdose of sodium pentobarbital administered IV or IC, followed by auscultation or palpation to ensure death. Ocular tissues were then dissected.

Blood Collections (for plasma)—New Zealand White Rabbits. At the timepoints depicted in the experimental design tables, approximately 3 mL of whole blood drawn from the marginal ear vein or cardiac puncture was collected in K₂EDTA tubes for plasma acquisition. After collection, the tubes were gently mixed and blood samples were stored on wet ice for up to 30 minutes prior to plasma processing. The samples were centrifuged at 4° C. for 10 minutes at 2000 g in a swinging bucket refrigerated centrifuge. Immediately after centrifugation, the clear plasma was transferred to 2 mL screw-cap polypropylene tubes and stored frozen at −80° C.

Blood Collections (for plasma)—Yucatan Minipigs. At the timepoints depicted in the experimental design table, approximately 1.5 mL (non-terminal timepoints) or at least 3 mL (terminal timepoint) of blood was drawn from the jugular, cephalic, or marginal ear vein, or via cardiac puncture (terminal timepoint only). Once drawn, blood was deposited into purple-topped K2EDTA tubes, which were gently mixed. Samples were stored on wet ice for up to 30 minutes prior to processing, then centrifuged at 4° C. for 10 minutes at 2000 g. Immediately after centrifugation, the clear plasma was transferred to a pre-labeled 2 mL polypropylene tube for each animal, then placed on dry ice prior to storing frozen at −80° C.

Ocular Tissue Dissections for Bioanalysis—New Zealand White Rabbits. Following euthanasia and confirmation of death, designated eyes were enucleated for bioanalysis. Aqueous humor was removed via a 27- or 30-G syringe and placed into a pre-weighed vial for each eye, which was weighed and snap frozen. Globes were then snap frozen in liquid nitrogen and stored at −80° C. until frozen dissection to prevent cross-contamination between tissue samples. Tissues were dissected and were stored in individual prelabeled, pre-weighed vials, which were then re-weighed. Cornea and sclera, following isolation, were cut into fine pieces with a scalpel blade prior to placing into sample tubes to facilitate homogenization. Samples were stored at −80° C.

### List of Tissues Collected:

Aqueous humor—2 mL MPI Lysing Matrix M homogenization tubes
Bulbar conjunctiva—2 mL MPI Lysing Matrix homogenization tubes
Cornea—2 mL MPI Lysing Matrix M homogenization tubes.
Sclera—2 mL MPI Lysing Matrix M homogenization tubes (split into 2 tubes if needed)
Iris/ciliary body—2 mL MPI Lysing Matrix M homogenization tubes
Vitreous humor—2 mL MPI Lysing Matrix M homogenization tubes (split into 2 tubes if needed)
Retina—2 mL MPI Lysing Matrix M homogenization tubes
RPE/choroid—2 mL MPI Lysing Matrix M homogenization tubes.

Ocular Collections for Pharmacokinetics—Yucatan Minipigs. Immediately following euthanasia, designated eyes were enucleated and aqueous humor was removed via a 27- or 30-gauge syringe. The bulbar conjunctiva was dissected, and the entire globe was snap frozen and placed into a polypropylene tube for storage at −80° C. until frozen ocular dissections were performed. Ocular dissections were performed with light thawing of the eye. Cornea and sclera were to be minced with a scalpel blade prior to placement in homogenization tubes in order to facilitate homogenization. Tissues were allowed to be split into 2-3 tubes as needed. The following samples were placed into vials.

List of tissues collected (individual tubes per animal eye):
Plasma—2 mL polypropylene tubes
Aqueous humor—2 mL polypropylene tubes
Bulbar conjunctiva—2 mL MPI Lysing Matrix M homogenization tubes
Vitreous humor—2 mL MPI Lysing Matrix M homogenization tubes
Sclera—2 mL MPI Lysing Matrix M homogenization tubes.
Cornea—2 mL MPI Lysing Matrix M homogenization tubes
Lens—2 mL MPI Lysing Matrix M homogenization tubes
Iris—Ciliary Body—2 mL MPI Lysing Matrix M homogenization tubes
Retina—2 mL MPI Lysing Matrix M homogenization tubes
RPE/choroid—2 mL MPI Lysing Matrix M homogenization tubes.

Bioanalysis of ocular and plasma samples. Plasma and eye tissue concentrations of bevemipretide were analyzed by LC-MS/MS (LLOQ: 0.1-1 ng/ml; 0.2-5 ng/g).

### Results:

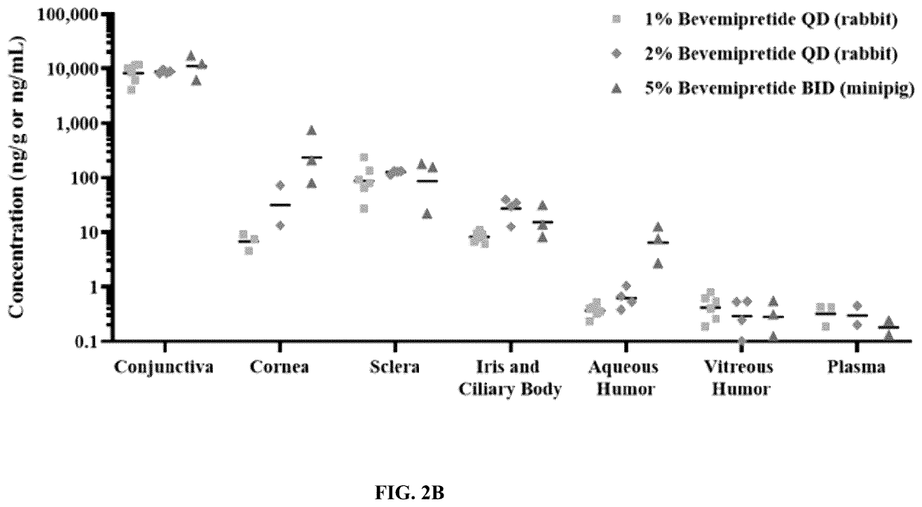

Tissue and plasma concentrations of bevemipretide 24 hours after final dose on Day 30 in New Zealand white rabbits (1% or 2% bevemipretide QD) and on Day 29 in Yucatan minipigs (5% BID) are shown in FIGS. 2A and 2B. These results demonstrate that there is suitable exposure of bevemipretide in retinal tissues when administered topically (i.e., via eye drops) with low systemic (as determined from plasma) exposure. In addition, based on ocular examination, topical administration of the bevemipretide was well-tolerated by the rabbits and minipigs at all concentrations (i.e., 1% QD, 2% QD, and 5% BID bevemipretide), and 1%, 2% and 3% (wt./vol.) bevemipretide topical drops were well tolerated in a P1 human clinical trial.

### Equivalents

The present technology is not to be limited in terms of the particular embodiments described in this application, which are intended as single illustrations of individual aspects of the present technology. Many modifications and variations of this technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that the present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for preventing, inhibiting, ameliorating, or delaying thinning of the retinal outer nuclear layer (ONL) in a subject in need thereof, the method comprising administering to the subject a therapeutically effective amount of bevemipretide, or a pharmaceutically acceptable salt, stereoisomer, tautomer, hydrate, and/or solvate thereof.

2. A method for preventing, inhibiting, ameliorating, or delaying reduction in total retinal thickness in a subject in need thereof, the method comprising administering to the subject a therapeutically effective amount of bevemipretide, or a pharmaceutically acceptable salt, stereoisomer, tautomer, hydrate, and/or solvate thereof.

3. A method for protecting the retinal outer nuclear layer (ONL) and/or the retina from light- or age-induced damage in a subject in need thereof, the method comprising administering to the subject a therapeutically effective amount of bevemipretide, or a pharmaceutically acceptable salt, stereoisomer, tautomer, hydrate, and/or solvate thereof.

4. A method for protecting photoreceptors from light- or age-induced damage in a subject in need thereof, the method comprising administering to the subject a therapeutically effective amount of bevemipretide, or a pharmaceutically acceptable salt, stereoisomer, tautomer, hydrate, and/or solvate thereof.

5. The method of claim 1, wherein the subject is a mammal.

6. The method of claim 5, wherein the subject is a human.

7. The method of claim 1, wherein the bevemipretide, or a pharmaceutically acceptable salt, stereoisomer, tautomer, hydrate, and/or solvate thereof is administered topically, subcutaneously, intraocularly, or ophthalmically.

8. The method of claim 1, wherein the bevemipretide, or a pharmaceutically acceptable salt, stereoisomer, tautomer, hydrate, and/or solvate thereof is administered orally, intranasally, systemically, intravenously, intraperitoneally, intradermally, intrathecally, intracerebroventricularly, iontophoretically, transmucosally, or intramuscularly.

9. The method of claim 1, wherein the bevemipretide, or a pharmaceutically acceptable salt, stereoisomer, tautomer, hydrate, and/or solvate thereof is administered daily for 2 weeks or more, 12 weeks or more, 24 weeks or more, 52 weeks or more, or 2 years or more.

10. The method of claim 1, wherein the bevemipretide, or a pharmaceutically acceptable salt, stereoisomer, tautomer, hydrate, and/or solvate thereof is administered via eye drops comprising the bevemipretide, or a pharmaceutically acceptable salt, stereoisomer, tautomer, hydrate, and/or solvate thereof.

11. The method of claim 10, wherein the eye drops comprise the bevemipretide, or a pharmaceutically acceptable salt, stereoisomer, tautomer, hydrate, and/or solvate thereof in a concentration of about 0.2% to about 5% (wt./vol.) inclusive, about 1% to about 5% (wt./vol.) inclusive, about 1% to about 3% (wt./vol.) inclusive or about 0.5% to about 2% (wt./vol.) inclusive.

12. The method of claim 10, wherein the eye drops comprise the bevemipretide, or a pharmaceutically acceptable salt, stereoisomer, tautomer, hydrate, and/or solvate thereof in a concentration of about 0.5% (wt./vol.), about 1% (wt./vol.), about 1.5% (wt./vol.), about 2% (wt./vol.), about 2.5% (wt./vol.), or about 3% (wt./vol.).

13. The method of claim 1, further comprising separately, sequentially, or simultaneously administering an additional treatment to the subject.

14. The method of claim 13, wherein the additional treatment comprises administering an AREDS or AREDS 2 vitamin formula to the subject.

15. The method of claim 13, wherein the additional treatment comprises administering an antioxidant, a metal complexer, an anti-inflammatory drug, an antibiotic, a mast cell stabilizer, and/or an antihistamine to the subject.

16. The method of claim 15, wherein the antioxidant is vitamin A, vitamin C, vitamin E, lycopene, selenium, α-lipoic acid, coenzyme Q, glutathione, or a carotenoid.

17. The method of claim 13, wherein the additional treatment comprises administering mometasone furoate, tacrolimus, quercetin, or diphenhydramine to the subject.

18. The method of claim 13, wherein the additional treatment comprises administering a flavonoid, a coumarin, a phenol or a terpenoid to the subject.

19. The method of claim 18, wherein the flavonoid is luteolin (3′,4′,5,7-tetrahydroxyflavone), diosmetin (5,7,3′-trihydroxy-4′-methoxyflavone), apigenin (4′,5,7-trihydroxyflavone), quercetin (3,3′,4′,5,7-pentahydroxyflavone), fisetin (2-(3,4-dihydroxyphenyl)-3,7-dihydroxychromen-4-one), kaempferol (3,4′,5,7-tetrahydroxyflavone), ginkgetin (7,4′-dimethylamentoflavone) or silymarin.

20. The method of claim 18, wherein the coumarin is scopletin (6-methoxy-7 hydroxycoumarin), scaporone (6,7-dimethoxycoumarin), artekeiskeanol A (7-{[(2E,6E)-8-Hydroxy-3,7-dimethylocta-2,6-dien-1-yl]oxy}-6-methoxy-2H-chromen-2-one), selinidin ((8,8-dimethyl-2-oxo-9,10-dihydropyrano[2,3-h]chromen-9-yl) 2-methylbut-2-enoate), 5-methoxy-8-(2-hydroxy-3-butoxy-3-methylbutyloxy)-psoralen, cinnamic acid ((2)-3-phenylprop-2-enoic acid) or ellagic acid (2,3,7,8-tetrahydroxy[1]benzopyrano[5,4,3-cde][1]benzopyran-5,10-dione).

21. The method of claim 18, wherein the phenol is magnolol (5,5′-di(prop-2-en-1-yl)[1,1′-biphenyl]-2,2′-diol), honokiol (3′,5-di(prop-2-en-1-yl)[1,1′-biphenyl]-2,4′-diol), resveratrol (5-[E-2-(4-hydroxyphenyl) ethen-1-yl]benzene-1,3-diol), polydatin (3,4′,5-trihydroxystilbene-3-β-d-glucoside), curcumin ((1E,6E)-1,7-bis(4-hydroxy-3-methoxyphenyl)hepta-1,6-diene-3,5-dione), α-mangostin (1,3,6-trihydroxy-7-methoxy-2,8-bis(3-methylbut-2-en-1-yl)-9H-xanthen-9-one), β-mangostin (1,6-dihydroxy-3,7-dimethoxy-2,8-bis(3-methylbut-2-enyl)xanthen-9-one) or γ-mangostin (1,3,6,7-tetrahydroxy-2,8-bis(3-methylbut-2-en-1-yl)-9H-xanthen-9-one).

22. The method of claim 18, wherein the terpenoid is parthenolide ((1aR,4E,7aS,10aS,10bR)-2,3,6,7,7a,8,10a,10b-octahydro-1a,5-dimethyl-8-methylene-oxireno[9,10]cyclodeca[1,2-b]furan-9 (1aH)-one), sinomenine, indoline (2,3-dihydro-1H-indole) or xestospongin C([1R-(1R,4aR,11R,12aS,13S,16aS,23R,24aS)]-eicosahydro-5H, 17H-1,23:11,13-diethano-2H, 14H-[1,11]dioxacycloeicosino[2,3-b:12,13-b1]dipyridine).

23. The method of claim 13, wherein the additional treatment comprises administering to the subject a therapeutic agent selected from the group consisting of: aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, pharmaceutically acceptable salts thereof, and combinations thereof.

24. The method of claim 1, wherein the pharmaceutically acceptable salt comprises a tartrate salt, a fumarate salt, monoacetate salt, a bis-acetate salt, a tri-acetate salt, a mono-trifluoroacetate salt, a bis-trifluoroacetate salt, a trifluoroacetate salt, a monohydrochloride salt, a bis-hydrochloride salt, a trihydrochloride salt, a mono-tosylate salt, a bis-tosylate salt, or a tri-tosylate salt.

25. The method of claim 1, wherein the bevemipretide administered is formulated from a tris-HCl salt, a bis-HCl salt, or a mono-HCl salt.

26. The method of claim 1, wherein bevemipretide administered is formulated from the tris-HCl salt of formula:

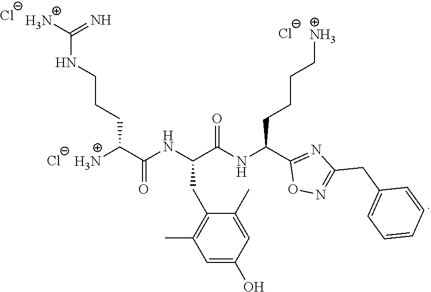 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

27. The method of claim 4, wherein the photoreceptors are protected from light-induced damage.

28. The method of claim 4, wherein the photoreceptors are protected from age-induced damage.
